(19) United States
(12) Patent Application Publication
(10) Pub. No.: US 2022/0289825 A1
(43) Pub. Date: Sep. 15, 2022

(54) FUSION PROTEIN TARGETING PD-L1 AND TGF-ß AND USE THEREOF

(71) Applicants: Nanjing Shunxin Pharmaceuticals Co., Ltd., Of Chiatai Tianqing Pharmaceutical Group, Nanjing, Jiangsu Province (CN); Chia Tai Tianqing Pharmaceutical Group Co., Ltd., Lianyungang, Jiangsu Province (CN)

(72) Inventors: Wei ZHAO, Lianyungang, Jiangsu Province (CN); Yingchun LI, Lianyungang, Jiangsu Province (CN); Lianxiang XIE, Lianyungang, Jiangsu Province (CN); Yamin LU, Lianyungang, Jiangsu Province (CN); Haili LV, Lianyungang, Jiangsu Province (CN); Xiuzhen DU, Lianyungang, Jiangsu Province (CN); Tongjie XU, Lianyungang, Jiangsu Province (CN); Yanju CHENG, Lianyungang, Jiangsu Province (CN); Xiquan ZHANG, Lianyungang, Jiangsu Province (CN)

(21) Appl. No.: 17/637,697

(22) PCT Filed: Aug. 28, 2020
(86) PCT No.: PCT/CN2020/111983

§ 371 Date: Feb. 23, 2022

(30) Foreign Application Priority Data
Aug. 30, 2019 (CN) 201910815301.2

(51) Int. Cl. C07K 14/71; A61P 35/00; C12N 15/63; C07K 14/705

(52) U.S. Cl. CPC C07K 14/71; A61P 35/00; C12N 15/63; C07K 14/70532; C07K 2317/92; C07K 2317/565; C07K 2317/76; A61K 38/00

(57) ABSTRACT

Provided are a fusion protein targeting PD-L1 and TGF-β and use thereof. The fusion protein is a fusion protein comprising an antibody or antigen-binding fragment that specifically binds to human PD-L1 and a human TGFβRII binding domain. Further provided are a polynucleotide encoding the fusion protein, a host cell containing the polynucleotide, and the use of the fusion protein in the preparation of an anti-tumor drug.

### FIELD OF THE INVENTION

The present application relates to the field of biotechnology, and specifically to a fusion protein comprising: (a) at least one heavy chain variable domain and at least one light chain variable domain of an antibody binding to human programmed death-ligand 1 (PD-L1); and (b) a human TGF-β RII or a TGF-β binding fragment thereof. The present application further relates to a method for preparing the fusion protein and use thereof (e.g., for cancer treatment).

### BACKGROUND

In recent years, tumors show increasing incidences, along with poor efficacy for malignancies and high metastasis rate in advanced diseases. At present, conventional therapies in the clinical use, such as radiotherapy, chemotherapy and surgery, can relieve the pain and prolong the survival period, but have limitations. Therefore, a method for activating the immune system and reforming the host's immune response by immunotherapy to induce tumor regression and stabilize the disease has gradually become a hot spot in the field of oncotherapy. Programmed death-ligand 1 (PD-L1), one of the ligands of programmed death receptor 1 (PD-1), is mainly expressed on T cells, B cells, macrophages and dendritic cells, and demonstrates up-regulated expression on activated cells. Binding of PD-L1 and PD-1 forms a receptor-ligand complex before sending inhibitory signals, including signals for inducing the IL-10 (inflammation and immunosuppressive factors) production, down-regulating anti-apoptosis gene bcl-2 to promote the apoptosis of antigen-specific T cells, inhibiting the proliferation of CD8⁺ T cells in lymph nodes, etc. In addition to the capability of binding to PD-1 with a high affinity, PD-L1 can also bind to CD80 with a low affinity, thereby suppressing T cell activity. Researches show that PD-L1 protein is highly expressed in various human tumor tissues, such as breast cancer, lung cancer, gastric cancer, intestinal cancer, kidney cancer and melanoma, such that tumor cells can evade the attack of the immune system. Meanwhile, the expression level of PD-L1 is closely related to the clinical treatment and prognosis of a subject. This suggests that PD-L1 is a prospective target in tumor immunotherapy.

Transforming growth factor β (TGF-β) is a multifunctional cytokine that plays an important regulatory role in cell proliferation and differentiation, migration and adhesion, extracellular matrix production, angiogenesis and connective tissue formation, immune functions, and other processes. TGF-β has both tumor-suppressing and tumor-promoting effects. However, with the progression of tumors, it promotes the tumor metastasis and immune escape as well as induces tumor angiogenesis by epithelial-mesenchymal transition, ultimately leading to disease progression. TGF-β recognizes transforming growth factor β receptor II (TGF-β RII) and subsequently forms a complex that phosphorylates the TGF-β RII. Then the complex binds to TGF-β RI dimer to form a heterotetrameric receptor complex for signaling. Researches show that inhibition of TGF-β signaling by TGF-β receptors can reduce tumor metastasis. This makes the TGF-β receptor one of the important directions for developing tumor drugs at present.

Inhibitors of PD-L1 and other immune checkpoint proteins act less efficiently. They have long-term effects only on certain subjects, and may lead to fatal autoimmune adverse events. Therefore, how to improve their therapeutic effects and reduce the toxicity remains a focus of research in the field of immunotherapy. However, recent researches showed that TGF-β is the leading cause of the failure of immune checkpoint inhibitor therapies. In general, inhibiting TGF-β signaling pathways, on the basis of inhibiting PD-L1/PD-1 pathways, can effectively improve the anti-tumor efficacy.

### SUMMARY

The purpose of the present application is at least to provide a fusion protein targeting PD-L1 and TGF-β, which may

a) specifically bind to PD-L1 and/or block the binding of PD-1/PD-L1 and a signaling pathway thereof; and/or

b) bind to TGF-β or inhibit TGF-β signaling, so as to reduce its promoting effect on tumor progression. In another aspect, the present application provides a protein molecule comprising a partial or intact TGF-β RII that can bind to TGF-β, and an antibody or an antigen binding fragment thereof that binds to an immune checkpoint protein (e.g., PD-L1), as an effective anti-tumor and anti-cancer medicament.

In yet another aspect, the present application provides a fusion protein comprising:

(a) an antibody or an antigen binding fragment thereof (e.g., at least one heavy chain variable domain and at least one light chain variable domain) that binds to human programmed death-ligand 1 (PD-L1); and/or

(b) a human TGF-β binding domain.

In certain embodiments, the human TGF-β binding domain is a human TGF-β RII or a TGF-β binding fragment thereof, e.g., a polypeptide or peptide fragment having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 36, or any fragment described herein.

In certain embodiments, a CDR1 sequence of the heavy chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 1 or SEQ ID NO: 15, a CDR2 sequence of the heavy chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 2 or SEQ ID NO: 16, and a CDR3 sequence of the heavy chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 3 or SEQ ID NO: 17; and a CDR1 sequence of the light chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 4 or SEQ ID NO: 18, a CDR2 sequence of the light chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 5 or SEQ ID NO: 19, and a CDR3 sequence of the light chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 6 or SEQ ID NO: 20. In certain embodiments, the heavy chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 7 or SEQ ID NO: 21, and the light chain variable domain has at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 8 or SEQ ID NO: 22.

HCDR1:
SEQ ID NO: 1
SYGMS
SEQ ID NO: 15
TYGVH
HCDR2:
SEQ ID NO: 2
SISSGGSTYYPDSVKG
SEQ ID NO: 16
VIWRGVTTDYNAAFMS
HCDR3:
SEQ ID NO: 3
GYDSGFAY
SEQ ID NO: 17
LGFYAMDY
LCDR1:
SEQ ID NO: 4
ASQSVSTSSSSFMH
SEQ ID NO: 18
KASQSVSNDVA
LCDR2:
SEQ ID NO: 5
YASNLES
SEQ ID NO: 19
YAANRYT
LCDR3:
SEQ ID NO: 6
QHSWEIPYT
SEQ ID NO: 20
QQDYTSPYT

The fusion protein may further comprise a linker peptide linking a C terminus of the antibody or the antigen binding fragment thereof to an N terminus of the TGF-β binding domain. In certain embodiments, the linker peptide may be a flexible linker peptide or a rigid linker peptide, and optionally is a combination of glycine and serine. For example, the linker peptide is (G₄S)ₓG, wherein x is optionally an integer of 3-6, preferably 4-5, and most preferably 4. Linkage without using a linker peptide can also be employed.

In certain embodiments, the fusion protein comprises at least one light chain variable domain and at least one heavy chain variable domain of an antibody. When combined, the light chain variable domain and the heavy chain variable domain form an antigen binding site that binds to human PD-L1.

In certain embodiments, the fusion protein may comprise a constant region of an immunoglobulin, or a fragment, analog, variant or derivative of the constant region. In some embodiments, the constant region is derived from a human immunoglobulin heavy chain, e.g., a heavy chain of IgG1, IgG2, IgG3 and IgG4, or other types of immunoglobulins, and preferably a heavy chain of IgG1. In some embodiments, the constant region may comprise any modification described herein, e.g., insertion, deletion, substitution, or chemical modification of amino acids. In some embodiments, the constant region comprises a mutation that alters the effector function. For example, a lysine residue at the C terminus of the antibody constant region is mutated to a hydrophobic amino acid, such as alanine or leucine, thereby reducing hydrolytic cleavage by proteases and prolonging the serum half-life. In some embodiments, any amino acid residue of the constant region may be substituted by an amino acid residue of any allotype, preferably by an amino acid residue of Glm(3) and/or nGlm(1).

In certain embodiments, the fusion protein comprises an antibody or an antigen binding fragment thereof that binds to PD-L1, and a TGF-β binding domain. The antibody or the antigen binding fragment thereof may optionally comprise a modified (e.g., any modification described herein) constant region, comprising a mutation of K at the C terminus of the constant region to A, or a substitution by an amino acid of an allotype. The TGF-β binding domain comprises a human TGF-β RII or a fragment or variant thereof that can bind to TGF-β, or an extracellular domain of human TGF-β RII.

In certain embodiments, the fusion protein comprises (a) a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a heavy chain variable domain amino acid sequence set forth in SEQ ID NO: 7 and a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a light chain variable domain amino acid sequence set forth in SEQ ID NO: 8, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a heavy chain variable domain amino acid sequence set forth in SEQ ID NO: 21 and a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a light chain variable domain amino acid sequence set forth in SEQ ID NO: 22; and (b) a TGF-β binding domain. In some certain embodiments, the TGF-β binding domain is a human TGF-β RII or a TGF-β binding fragment or a variant thereof, or a polypeptide or peptide fragment having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 36:

(SEQ ID NO: 7)
EVQLVESGGGLVKPGGSLRLSCAASGFIFRSYGMSWVRQAPGKGLEWVAS
ISSGGSTYYPDSVKGRFTISRDNAKNSLYLQMNSLRAEDTAVYDCARGYD
SGFAYWGQGTLVTVSS;
(SEQ ID NO: 8)
DIVLTQSPASLAVSPGQRATITCRASQSVSTSSSSFMHWYQQKPGQPPKL
LIKYASNLESGVPARFSGSGSGTDFTLTINPVEANDTANYYCQHSWEIPY
TFGQGTKLEIK;
(SEQ ID NO: 21)
QITLKESGPTLVKPTQTLTLTCTVSGFSLSTYGVHWIRQPPGKALEWLGV
IWRGVTTDYNAAFMSRLTITKDNSKNQVVLTMNNMDPVDTATYYCARLGF
YAMDYWGQGTLVTVSS;
(SEQ ID NO: 22)
DIQMTQSPSSLSASVGDRVTITCKASQSVSNDVAWYQQKPGKAPKLLIYY
AANRYTGVPDRFSGSGYGTDFTFTISSLQPEDIATYFCQQDYTSPYTFGQ
GTKLEIK;
(SEQ ID NO: 36)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD.

In certain embodiments, the fusion protein comprises (a) a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a heavy chain amino acid sequence set forth in SEQ ID NO: 9 and a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a light chain amino acid sequence set forth in SEQ ID NO: 10, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a heavy chain amino acid sequence set forth in SEQ ID NO: 23 and a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a light chain amino acid sequence set forth in SEQ ID NO: 24; and (b) a TGF-β binding domain. In certain embodiments, the fusion protein comprises (a) a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a heavy chain amino acid sequence set forth in SEQ ID NO: 29 and a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a light chain amino acid sequence set forth in SEQ ID NO: 10, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a heavy chain amino acid sequence set forth in SEQ ID NO: 32 and a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to a light chain amino acid sequence set forth in SEQ ID NO: 24; and (b) a TGF-β binding domain. In some certain embodiments, the TGF-β binding domain is a human TGF-β RII or a TGF-β binding fragment or a variant thereof, or a polypeptide or peptide fragment having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 36.

In some embodiments, the fusion protein comprises any antigen binding protein described in the art that specifically binds to PD-L1 or an antigen binding fragment thereof. Preferably, the antigen binding protein that specifically binds to PD-L1 comprises the following amino acid sequences: a heavy chain CDR1 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 1 or SEQ ID NO: 15; a heavy chain CDR2 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 2 or SEQ ID NO: 16; a heavy chain CDR3 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 3 or SEQ ID NO: 17; a light chain CDR1 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 4 or SEQ ID NO: 18; a light chain CDR2 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 5 or SEQ ID NO: 19; and a light chain CDR3 having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 6 or SEQ ID NO: 20.

In some embodiments, the antigen binding protein that specifically binds to PD-L1 comprises the following amino acid sequences: a heavy chain CDR1 selected from SEQ ID NO: 1 and SEQ ID NO: 15; a heavy chain CDR2 selected from SEQ ID NO: 2 and SEQ ID NO: 16; a heavy chain CDR3 selected from SEQ ID NO: 3 and SEQ ID NO: 17; a light chain CDR1 selected from SEQ ID NO: 4 and SEQ ID NO: 18; a light chain CDR2 selected from SEQ ID NO: 5 and SEQ ID NO: 19; and a light chain CDR3 selected from SEQ ID NO: 6 and SEQ ID NO: 20.

In some embodiments, the antigen binding protein that specifically binds to PD-L1 comprises the following amino acid sequences: a heavy chain variable domain having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 7 or SEQ ID NO: 21; and a light chain variable domain having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 8 or SEQ ID NO: 22.

In some embodiments, the antigen binding protein that specifically binds to PD-L1 comprises the following amino acid sequences: a heavy chain variable domain as set forth in SEQ ID NO: 7; and a light chain variable domain as set forth in SEQ ID NO: 8.

In some embodiments, the antigen binding protein that specifically binds to PD-L1 comprises the following amino acid sequences: a heavy chain variable domain as set forth in SEQ ID NO: 21; and a light chain variable domain as set forth in SEQ ID NO: 22.

Preferably, the antigen binding protein that specifically binds to PD-L1 comprises the following amino acid sequences: a heavy chain amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 9 or SEQ ID NO: 23, or a heavy chain amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 29 or SEQ ID NO: 32; and a light chain amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 10 or SEQ ID NO: 24.

Preferably, the antigen binding protein that specifically binds to PD-L1 comprises the following amino acid sequences: a heavy chain amino acid sequence as set forth in SEQ ID NO: 9; and a light chain amino acid sequence as set forth in SEQ ID NO: 10.

Preferably, the antigen binding protein that specifically binds to PD-L1 comprises the following amino acid sequences: a heavy chain amino acid sequence as set forth in SEQ ID NO: 23; and a light chain amino acid sequence as set forth in SEQ ID NO: 24.

Preferably, the antigen binding protein that specifically binds to PD-L1 comprises the following amino acid sequences: a heavy chain amino acid sequence as set forth in SEQ ID NO: 29; and a light chain amino acid sequence as set forth in SEQ ID NO: 10.

Preferably, the antigen binding protein that specifically binds to PD-L1 comprises the following amino acid sequences: a heavy chain amino acid sequence as set forth in SEQ ID NO: 32; and a light chain amino acid sequence as set forth in SEQ ID NO: 24.

In certain embodiments, the fusion protein comprises: (1) two identical first polypeptides with an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 11 or SEQ ID NO: 25, or with an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 30 or SEQ ID NO: 33; and (2) two identical second polypeptides with an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to an amino acid sequence set forth in SEQ ID NO: 13 or SEQ ID NO: 27. Wherein, the amino acid sequence of the first polypeptide sequentially comprises, from the N terminus to the C terminus, the following sequences: a heavy chain variable domain of an antibody that recognizes PD-L1 antigenic epitope or antigen, an antibody constant region, a linker peptide, and a TGF-β binding fragment of a human TGF-β RII.

In another aspect, the present application further provides a polynucleotide sequence encoding the fusion protein described above. For example, the polynucleotide sequence comprises: a nucleic acid sequence of SEQ ID NO: 12 encoding an amino acid sequence set forth in SEQ ID NO: 11, a nucleic acid sequence of SEQ ID NO: 26 encoding an amino acid sequence set forth in SEQ ID NO: 25, a nucleic acid sequence of SEQ ID NO: 31 encoding an amino acid sequence set forth in SEQ ID NO: 30, or a nucleic acid sequence of SEQ ID NO: 34 encoding an amino acid sequence set forth in SEQ ID NO: 33; and a nucleic acid sequence of SEQ ID NO: 14 encoding an amino acid sequence set forth in SEQ ID NO: 13, or a nucleic acid sequence of SEQ ID NO: 28 encoding an amino acid sequence set forth in SEQ ID NO:27. Preferably, the polynucleotide sequence comprises nucleic acid sequences set forth in SEQ ID NO: 34 and SEQ ID NO: 28, or nucleic acid sequences set forth in SEQ ID NO: 31 and SEQ ID NO: 14. The present application further provides an expression vector comprising the polynucleotide sequence. Alternatively, the present application further provides a cell comprising the polynucleotide sequence or the expression vector.

In some aspects, the present application further relates to a pharmaceutical composition comprising the fusion protein.

In yet another aspect, the present application further provides a method for producing the fusion protein. The fusion protein comprises: (a) at least one heavy chain variable domain and at least one light chain variable domain of an antibody binding to human programmed death-ligand 1 (PD-L1); and (b) a human TGF-β RII or a TGF-β binding fragment or a variant thereof. The method comprises: preparing a recombinant DNA by gene recombination technology, introducing the recombinant DNA into a cell, and allowing the cell to stably express the protein. The cell can be selected from the group consisting of a bacterium, a yeast and a mammalian cell, and preferably a mammalian cell, e.g., CHO, NSO, COS and BHK cells. The method further comprises harvesting a culture of the cell and purifying the obtained protein.

The present application further provides use of the fusion protein or protein molecule in preparing drugs for treating cancer, inhibiting tumor growth or enhancing anti-tumor response. The treatment is selected based on factors such as the sensitivity of the subject to the therapy targeting PD-L1 and TGF-β, clinical experience, and the expression levels of PD-L1 and TGF-β in the subject. The present application further relates to a fusion protein for use in treating cancer, inhibiting tumor growth, or enhancing anti-tumor response.

In some aspects, the protein molecule or fusion protein described herein may be used for local depletion of TGF-β at a tumor site, and/or for blocking the signaling pathway of TGF-β in a cell (e.g., a tumor cell or an immune cell).

In some aspects, the protein molecule or the fusion protein described herein can be used for blocking the PD-L1 pathway, and/or promoting the killing of tumor cells by immune cells.

In some aspects, the protein molecule or the fusion protein described herein has use in treating cancer, inhibiting tumor growth, or enhancing anti-tumor response. The cancer or tumor includes, but is not limited to, lung adenocarcinoma, mucinous adenocarcinoma, low-grade brain neuroglioma, glioblastoma multiforme, mesothelioma, melanoma, thyroid cancer, renal cancer, liver cancer, acute myeloid leukemia, esophageal adenocarcinoma, lymphoma, non-small cell lung cancer, metastatic non-small cell lung cancer, advanced or recurrent non-small cell lung cancer, refractory non-small cell lung cancer after chemotherapy, metastatic non-squamous non-small cell lung cancer, advanced or recurrent non-squamous non-small cell lung cancer, unresectable advanced non-small cell lung cancer, occult non-small cell lung cancer, breast cancer, metastatic breast cancer, triple-negative breast cancer, advanced or recurrent breast cancer, locally recurrent breast cancer, inflammatory breast cancer, pancreatic ductal adenocarcinoma, metastatic pancreatic cancer, locally advanced unresectable pancreatic cancer, recurrent pancreatic cancer, prostate cancer, advanced or metastatic prostate cancer, locally advanced prostate cancer, castration-resistant prostate cancer, recurrent prostate cancer after castration, localized prostate cancer, progressive prostate cancer, colorectal cancer, rectal adenocarcinoma, large intestinal cancer, metastatic colorectal cancer, advanced or recurrent colon cancer, advanced or recurrent rectal cancer, locally recurrent rectal cancer, locally recurrent colon cancer, gastric adenocarcinoma, gastric cancer, unresectable gastric cancer, metastatic gastric cancer, locally advanced or recurrent gastric cancer, gastrointestinal stromal tumor, biliary tract cancer, bile duct cancer, gallbladder cancer, unresectable or metastatic biliary tract cancer, unresectable or metastatic bile duct cancer, unresectable or metastatic gallbladder cancer, penile cancer, anal cancer, vaginal cancer, cervical cancer, locally advanced cervical cancer, recurrent cervical cancer, metastatic cervical cancer, metastatic penile cancer, advanced or recurrent vaginal squamous cell carcinoma, advanced or recurrent vaginal adenocarcinoma, uterine corpus endometrioid carcinoma, bladder cancer, human papillomavirus infection, head and neck cancer, recurrent or metastatic head and neck cancer, hypopharyngeal cancer, laryngeal cancer, oral cancer, nasopharyngeal carcinoma, oropharyngeal cancer, pharyngolaryngeal cancer, paranasal sinus and nasal cavity cancer, and salivary gland cancer. The use may be to administer the protein molecule or the fusion protein, or a pharmaceutical composition comprising the protein molecule or the fusion protein, alone or in combination with other tumor therapies, such as radiotherapy, chemotherapy, surgery, biologics, and chemicals.

The present application further provides a method for treating cancer, inhibiting tumor growth, or enhancing anti-tumor response. The method comprises administering the protein molecule or the fusion protein, or a pharmaceutical composition comprising the protein molecule or the fusion protein, alone or in combination with other tumor therapies, such as radiotherapy, chemotherapy, surgery, biologics, and chemicals. The tumor or cancer includes, but is not limited to, lung adenocarcinoma, mucinous adenocarcinoma, low-grade brain neuroglioma, glioblastoma multiforme, mesothelioma, melanoma, thyroid cancer, renal cancer, liver cancer, acute myeloid leukemia, esophageal adenocarcinoma, lymphoma, non-small cell lung cancer, metastatic non-small cell lung cancer, advanced or recurrent non-small cell lung cancer, refractory non-small cell lung cancer after chemotherapy, metastatic non-squamous non-small cell lung cancer, advanced or recurrent non-squamous non-small cell lung cancer, unresectable advanced non-small cell lung cancer, occult non-small cell lung cancer, breast cancer, metastatic breast cancer, triple-negative breast cancer, advanced or recurrent breast cancer, locally recurrent breast cancer, inflammatory breast cancer, pancreatic ductal adenocarcinoma, metastatic pancreatic cancer, locally advanced unresectable pancreatic cancer, recurrent pancreatic cancer, prostate cancer, advanced or metastatic prostate cancer, locally advanced prostate cancer, castration-resistant prostate cancer, recurrent prostate cancer after castration, localized prostate cancer, progressive prostate cancer, colorectal cancer, rectal adenocarcinoma, large intestinal cancer, metastatic colorectal cancer, advanced or recurrent colon cancer, advanced or recurrent rectal cancer, locally recurrent rectal cancer, locally recurrent colon cancer, gastric adenocarcinoma, gastric cancer, unresectable gastric cancer, metastatic gastric cancer, locally advanced or recurrent gastric cancer, gastrointestinal stromal tumor, biliary tract cancer, bile duct cancer, gallbladder cancer, unresectable or metastatic biliary tract cancer, unresectable or metastatic bile duct cancer, unresectable or metastatic gallbladder cancer, penile cancer, anal cancer, vaginal cancer, cervical cancer, locally advanced cervical cancer, recurrent cervical cancer, metastatic cervical cancer, metastatic penile cancer, advanced or recurrent vaginal squamous cell carcinoma, advanced or recurrent vaginal adenocarcinoma, uterine corpus endometrioid carcinoma, bladder cancer, human papillomavirus infection, head and neck cancer, recurrent or metastatic head and neck cancer, hypopharyngeal cancer, laryngeal cancer, oral cancer, nasopharyngeal carcinoma, oropharyngeal cancer, pharyngolaryngeal cancer, paranasal sinus and nasal cavity cancer, and salivary gland cancer.

### Definitions

Unless otherwise specified, technical and scientific terms used herein have the same meaning as generally understood by those of ordinary skills in the art. If there are multiple definitions for a term in the present application, the definition used in this section shall be adopted unless otherwise specified.

The term “protein molecule” is sometimes referred to as the fusion protein herein.

The term “TGF-β RII” or “TGF-β receptor II” refers to a polypeptide or protein having a wild-type human TGF-β receptor type 2 isotype B sequence, e.g., a polypeptide set forth in SEQ ID NO: 35, or a polypeptide having a sequence substantially identical to an amino acid sequence set forth in SEQ ID NO: 35.

The term “fragment that binds to TGF-β” or “TGF-β binding fragment” of TGF-β RII refers to a fragment of TGF-β RII that has TGF-β binding activity and accounts for about at least 0.1%, 0.5%, 1%, 5%, 10%, 25%, 35%, 50%, 75%, 90%, 95%, 99% or 100% of the TGF-β RII sequence. The fragment is generally a soluble fragment, e.g., an extracellular domain of a human TGF-β RII or a variant thereof.

As used herein, the term “antibody” refers to a binding protein having at least one antigen binding domain. The antibody and the fragment thereof of the present application can be an intact antibody or any fragment thereof. Thus, the antibody and the fragment thereof of the present application include a monoclonal antibody or a fragment thereof and an antibody variant or a fragment thereof, as well as an immunoconjugate. Examples of the antibody fragment include a Fab fragment, a Fab′ fragment, a F(ab)′₂ fragments, a Fv fragment, a Fd′ fragment, an isolated CDR, a single-chain Fv molecule (scFv), and other antibody fragments known in the art, as well as antibodies with any modification known in the art (e.g., glycosylation, chemical modification, and the like). The antibody and the fragment thereof may also include a recombinant polypeptide, a fusion protein, and a bispecific antibody. The anti-PD-L1 antibody and the fragment thereof disclosed herein can be of IgG1, IgG2, IgG3, or IgG4 isotype. The term “isotype” refers to the class of antibodies encoded by the heavy chain constant region gene. The antibody and the fragment thereof may be a chimeric antibody, a humanized antibody or an intact human antibody.

The term “chimeric antibody” refers to an antibody having at least a portion of a heavy chain variable domain and a portion of a light chain variable domain derived from one species, and at least a portion of a constant region derived from another species. For example, in one embodiment, the chimeric antibody may comprise murine variable domains and a human constant region.

The term “humanized antibody” refers to an antibody comprising complementarity determining regions (CDRs) derived from a non-human antibody, and framework and constant regions derived from a human antibody. For example, the anti-PD-L1 antibody may comprise CDRs derived from one or more murine antibodies and human framework and constant regions. Exemplary humanized antibodies are disclosed herein. Additional anti-PD-L1 antibodies or variants thereof comprising the heavy and light chain CDRs disclosed herein can be generated using any human framework sequences, and are also included in the present application. In one embodiment, framework sequences suitable for use in the present application include those similar in structure to the framework sequences disclosed herein. Additional modifications may be made in the framework regions to improve the properties of the antibodies disclosed herein. Such additional framework modifications may include: chemical modifications, point mutations for reducing immunogenicity or removing T cell epitopes, or modifications reverting the mutations to residues in original germline sequences. In some embodiments, such modifications include those corresponding to the mutations exemplified herein, including reversions to germline sequences. For example, in one embodiment, one or more amino acids in the human VH and/or VL framework regions of the humanized antibodies disclosed herein are reverted to the corresponding amino acids in the parent murine antibodies.

As used herein, the term “derived”, when used to refer to a molecule or polypeptide relative to a reference antibody or other binding proteins, means a molecule or polypeptide that can specifically bind to the same epitope as the reference antibody or other binding proteins.

As used herein, the term “EC₅₀” refers to the effective concentration, 50% of the maximal response of an antibody. As used herein, the term “IC₅₀” refers to the inhibitory concentration, 50% of the maximal response of an antibody. Both EC₅₀ and IC₅₀ can be measured by ELISA or FACS assay or any other method known in the art.

The term “antigen binding fragment” refers to a fragment that retains the antigen binding function of a full-length antibody, including Fab, Fab′, F(ab′)₂, scFv, Fv, Fd, Fd′, an isolated CDR, a single-domain VHH fragment, and other antibody fragments known in the art, or fragments obtained by making any modification known in the art to the above fragments.

The term “linker peptide” refers to a polypeptide or peptide segment, preferably having synthetic amino acid sequences, that links the C terminus of an antibody or an antigen binding fragment thereof to the N terminus of the TGF-β binding domain in the fusion protein. The linker peptide used herein may be selected from (G₄S)ₓG, wherein x is optionally an integer of 3-6, preferably 4-5, and most preferably 4.

The term “identity” refers to the similarity between two reference sequences. The percent identity refers to a percentage that describes how similar sequences or designated regions of the sequences are by comparison using a sequence comparison algorithm known to those skilled in the art.

The term “substantially identical” refers to that the sequences have at least about 80% and higher (e.g., 81%, 82%, 83%, 84%, 85%, 86%, 87%, 88%, 89%, 90%, 91%, 92%, 93%, 94%, 95%, 96%, 97%, 98%, 99%, or 100%) identity.

The term “subject” refers to a mammal, including a human and a non-human animal, and preferably a human.

The term “treating” includes therapeutic treatment and prophylactic treatment or preventative measures, in which a therapeutic agent is administered to the subject to reduce at least one symptom of a disease, disorder, or condition (e.g., cancer or tumor), or to relieve the symptoms.

The term “cancer” refers to a collection of cells that proliferate in an abnormal manner.

Unless otherwise specified, terms in the singular shall be deemed to include the plural and vice versa. Unless otherwise specified, the word “a” or “an” refers to “at least one”. Unless otherwise stated, use of “or” means “and/or”.

Unless otherwise specified, the term “about” described herein refers to a fluctuation within ±5%, preferably within ±2%, and more preferably within ±1%, of the specified numerical range given.

As described herein, any percentage range, ratio range, or integer range shall be understood as including the value of any integer within the listed range and including, when appropriate, fractions thereof (such as one tenth and one hundredth of the integer) unless otherwise indicated.

As used herein, the terms “comprise”, “comprises” and “comprising” or equivalents thereof are open-ended statements and mean that elements, components and steps that are not specified may be included in addition to those listed, unless otherwise stated.

All patents, patent applications and other identified publications are expressly incorporated herein by reference for the purpose of description and disclosure. These publications are provided solely because they were disclosed prior to the filing date of the present application. All statements as to the dates of these documents or description as to the contents of these documents are based on the information available to the applicant and do not constitute any admission as to the correctness of the dates or the content of these documents. Moreover, in any country or region, any reference to these publications herein is not to be construed as an admission that the publications form part of the commonly recognized knowledge in the art.

### BRIEF DESCRIPTION OF THE DRAWINGS

FIG. 1 is a structural schematic of an anti-PD-L1-TGF-β RII fusion protein, in which the C terminus of the heavy chain of the anti-PD-L1 antibody is linked to the N terminus of the TGF-β receptor II extracellular domain by a (G₄S)₄G linker peptide.

FIG. 2A shows the biological activity of the PD-L1 terminus of hu5G11-hIgG1-TGF-β RII detected by reporter gene assay; FIG. 2B shows the biological activity of the PD-L1 terminus of M7824 detected by reporter gene assay.

FIG. 3 shows the in vitro binding activity of hu5G11-hIgG1-TGF-β RII to PD-L1 detected by ELISA assay.

FIG. 4 shows the in vitro binding activity of hu5G11-hIgG1-TGF-β RII to TGF-β1 detected by ELISA assay.

### EXAMPLES

The present application is further described below with reference to specific examples, which, however, are only for illustration and do not limit the scope of the present application. Likewise, the present application is not limited to any specific preferred embodiment described herein. It should be appreciated by those skilled in the art that equivalent substitutions or corresponding modifications made to the technical features of the present application still fall within the scope of the present application. Unless otherwise stated, the reagents used in the following examples are commercially available products, and the solutions can be prepared by conventional techniques in the art.

### Example 1 Construction and Expression of Fusion Protein Transient Expression Plasmid

An anti-PD-L1-TGF-β RII fusion protein hu5G11-hIgG1-TGF-β RII was constructed, as shown in FIG. 1. The light chain of this molecule had an amino acid sequence of SEQ ID NO: 27. The heavy chain (SEQ ID NO: 33) of this molecule was a fusion protein comprising a heavy chain of an anti-human-PD-L1 antibody (SEQ ID NO: 32), an amino acid sequence (SEQ ID NO: 36) of a human TGF-β RII extracellular domain, and a linker peptide (G₄S)₄G (SEQ ID NO: 37) linking the former two.

The light chain amino acid sequence (SEQ ID NO:
27) of the hu5G11-hIgGl-TGF-β RII was:
DIQMTQSPSSLSASVGDRVTITCKASQSVSNDVAWYQQKPGKAPKLLIYY
AANRYTGVPDRFSGSGYGTDFTFTISSLQPEDIATYFCQQDYTSPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
The heavy chain amino acid sequence (SEQ ID NO:
33) of the hu5G11-hIgGl-TGF-β RII was:
QITLKESGPTLVKPTQTLTLTCTVSGFSLSTYGVHWIRQPPGKALEWLGV
IWRGVTTDYNAAFMSRLTITKDNSKNQVVLTMNNMDPVDTATYYCARLGF
YAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVAVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGAGGGS
GGGGSGGGGSGGGGSGIPPHVQKSVNNDMIVTDNNGAVKFPQLCKFCDVR
FSTCDNQKSCMSNCSITSICEKPQEVCVAVWRKNDENITLETVCHDPKLP
YHDFILEDAASPKCIMKEKKKPGETFFMCSCSSDECNDNIIFSEEYNTSN
PD
The heavy chain amino acid sequence (SEQ ID NO:
32) of the PD-L1 antibody was:
QITLKESGPTLVKPTQTLTLTCTVSGFSLSTYGVHWIRQPPGKALEWLGV
IWRGVTTDYNAAFMSRLTITKDNSKNQVVLTMNNMDPVDTATYYCARLGF
YAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVAVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGA
The human TGF-β RII extracellular domain amino
acid sequence (SEQ ID NO: 36) was:
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD
The amino acid sequence (SEQ ID NO: 37) of the
linker peptide was:
GGGGSGGGGSGGGGSGGGGSG

A whole gene sequence containing the heavy chain nucleic acid sequence (SEQ ID NO: 34) and the light chain nucleic acid sequence (SEQ ID NO: 28) of the anti-PD-L1y-TGF-β RII fusion protein was synthesized and then incorporated to a eukaryotic expression vector, preferably a pcDNA3.1(+) expression vector, to construct the target expression plasmid. The plasmid was extracted to prepare the target plasmid. The expression plasmids for heavy and light chains were introduced into ExpiCHO-S™ cells using the ExpiFectamine™ CHO transfection technique for transient expression of the target protein. The obtained cell culture supernatant was purified to give the protein.

### Example 2 Isolation, Purification and Identification of Fusion Protein

The cell culture supernatant obtained in Example 1 was loaded onto a protein A affinity chromatography column, such as MabSelect from the GE company. The chromatography column was equilibrated with a phosphate equilibration buffer (e.g., 10 mM phosphate buffer, pH=6.0), and rinsed with a rinse buffer containing sodium chloride (e.g., a 25 mM phosphate buffer containing 0.5 M sodium chloride, pH=7.0-7.4) to remove partially bound impurities. Finally, the target protein product bound to the chromatography column were eluted with an eluent buffer to provide the anti-PD-L1-TGF-β RII fusion protein (i.e., hu5G11-hIgG1-TGF-β RII). The elution can be carried out by conventional methods, for example, using high-salt buffer or changing pH, such as using 1 M arginine hydrochloride buffer (pH=3-4) or 50 mM citrate buffer (pH=3-4) for elution. The phosphate buffer was prepared from disodium hydrogen phosphate and sodium dihydrogen phosphate, and the citrate buffer was prepared from citric acid and trisodium citrate. The eluate was quantified by UV analysis.

### Example 3 In-Vitro Binding Affinity Assay of Fusion Protein

By using Biacore T200, the affinities of the fusion protein hu5G11-hIgG1-TGF-β RII and M7824 to PD-L1 and TGF-β were analyzed. M7824 is the anti-PD-L1/TGF-β trap in Patent No. CN106103488, and the heavy and light chain sequences are set forth in SEQ ID NOs: 42 and 43 of the present application, respectively. The DNA sequences encoding heavy and light chains were cloned into expression vectors after synthesis. The light and heavy chain expression vectors were co-transfected into ExpiCHO-S™. The cells were incubated in an incubator at 37° C. with 8% CO₂. The culture supernatant was purified using a protein A filler as described in Example 2 to give M7824.

1) Affinity assay of the fusion protein to PD-L1: An anti-hIgG1(Fc) antibody was immobilized on the surface of a CM5 chip by amino coupling in an amount of about 8000-9000 RU. The fusion protein hu5G11-hIgG1-TGF-β RII and M7824 were captured using the CM5 chip. After diluting the PD-L1 protein (Sinobiological, 10084-H08H) using a running buffer to 20 nM, 10 nM, 5 nM, 2.5 nM and 1.25 nM, signals of interactions of the different concentrations of PD-L1 protein and the blank control (the running buffer) with the fusion protein hu5G11-hIgG1-TGF-β RII and M7824 were assayed, so as to obtain association and dissociation curves, The chip was regenerated with 3 mol/L MgCl₂ buffer to the baseline.

2) Affinity assay of the fusion protein to TGF-β: An anti-hIgG1(Fc) antibody was immobilized on the surface of a CM5 chip by amino coupling in an amount of about 8000-9000 RU. The fusion protein hu5G11-hIgG1-TGF-β RII and M7824 were captured using the CM5 chip. After diluting the TGF-β protein (Sinobiological, 10804-H08H) using a running buffer to 1000 nM, 500 nM, 250 nM, 125 nM and 62.5 nM, signals of interactions of the different concentrations of TGF-β protein and the blank control (the running buffer) with the fusion protein hu5G11-hIgG1-TGF-β RII and M7824 were assayed, so as to obtain association and dissociation curves, The chip was regenerated with 3 mol/L MgCl₂ buffer to the baseline.

The data signals were collected by BiaControl Software 2.0 in real time, and analyzed by BiaEvaluation Software 2.0. The data were fitted using a Langmuir 1:1 model after being subtracted (i.e., blank control signals were subtracted from sample signals in each cycle), so as to calculate the association rate constant Ka, the dissociation rate constant Kd and the equilibrium constant KD.

TABLE 1
Affinity of test samples to PD-L1 and TGF-β

| | | | |
|---|---|---|---|
| Sample | KD (M) | ka(1/Ms) | kd(1/s) |

PD-L1 terminus

| | | | |
|---|---|---|---|
| hu5G11-hIgG1-TGFβ RII | 4.27E−11 | 1.74E+07 | 7.45E−04 |
| M7824 | 7.37E−11 | 5.15E+07 | 3.79E−03 |

TGF-β terminus

| | | | |
|---|---|---|---|
| hu5G11-hIgG1-TGFβ RII | 1.13E−08 | 8.28E+04 | 9.37E−04 |
| M7824 | 1.52E−08 | 2.44E+05 | 3.71E−03 |

As can be seen from the above table, the fusion protein of the present application, as compared to the reference M7824, has smaller KD and Kd values, i.e., the fusion protein of the present application has higher affinity to both PD-L1 and TGF-β.

### Example 4 Biological Activity Assay of PD-L1 Terminus of hu5G11-hIgG1-TGF-β RII by Reporter Gene Assay

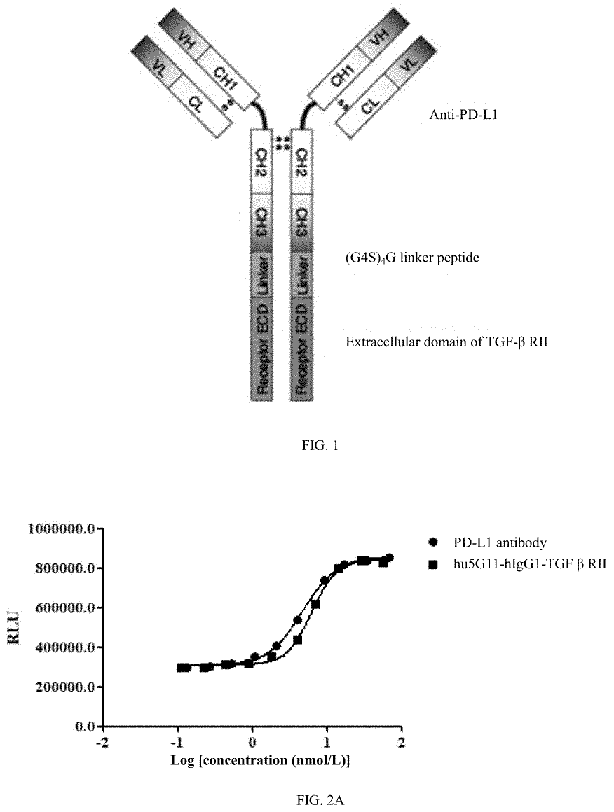

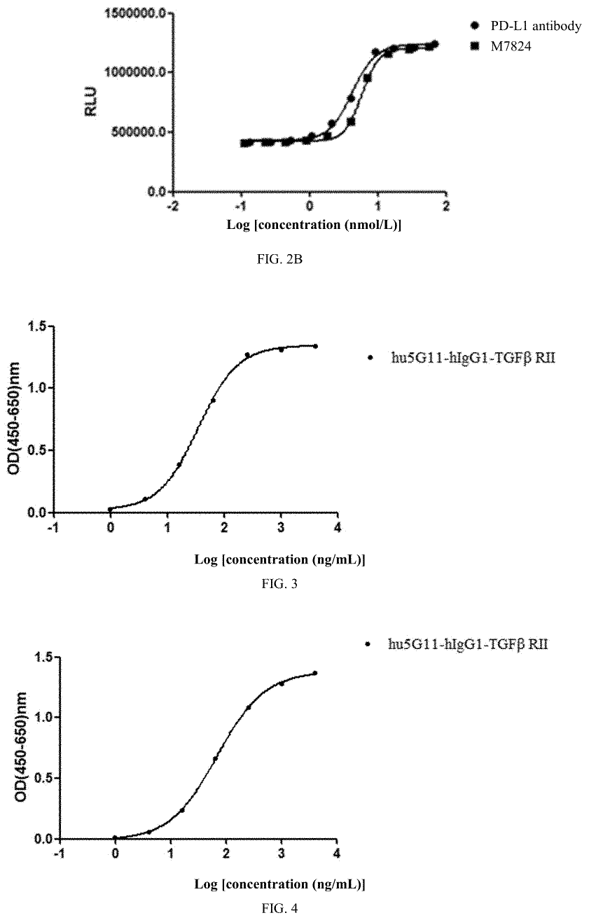

CHO-PDL1-CD3L cells (National Institutes for Food and Drug Control) were taken, adjusted to a viable cell density of 4-5×10⁵ cells/mL using a CHO-PDL1-CD3L complete medium, added to a white 96-well cell plate at 100 μL/well, and incubated in a cell incubator at 37° C. with 5% CO₂ for 16-20 h. Jurkat-PD-1-NFAT cells (National Institutes for Food and Drug Control) in the logarithmic growth phase were taken, and adjusted to a viable cell density of 1.25-2×10⁶ cells/mL using an assay medium. The cell plate was taken out, and the supernatant was discarded. The Jurkat-PD-1-NFAT cell suspension was added to the above cell plate at 50 μL/well. hu5G11-hIgG1-TGF-β RII was pre-diluted to a concentration of 56 nmol/L and then serially 2-fold diluted to the 10^{th} concentration (i.e., about 56 nmol/L, about 28 nmol/L, about 14 nmol/L, about 7 nmol/L, about 3.5 nmol/L, about 1.75 nmol/L, about 0.875 nmol/L, about 0.4375 nmol/L, about 0.21875 nmol/L, and about 0.109375 nmol/L). The PD-L1 antibody was pre-diluted to a concentration of 68 nmol/L and then serially 2-fold diluted to the 10^{th} concentration. M7824 was pre-diluted to a concentration of 56 nmol/L and then serially 2-fold diluted to the 10^{th} concentration. The serially diluted hu5G11-hIgG1-TGF-β RII, PD-L1 antibody and M7824 were added to the cell plate at 50 μL/well, and incubated in a cell incubator with 5% CO₂ at 37° C. for 6 h. Bio-Glo Luciferase reagent (Promega, G7940) was added to the above cell plate at 100 μL/well, and the cells were incubated in the dark at room temperature for 2-3 min. Relative light unit (RLU) was read by a multifunctional microplate reader (Thermo, Varioskan Flash). The biological activity results of the PD-L1 terminus of the fusion protein hu5G11-hIgG1-TGF-β RII described in the examples of the present application are shown in FIG. 2A and Table 2. The results in Table 2 indicate that the PD-L1 terminus of the fusion protein of the present application has higher biological activity.

Biological activity (%) of test sample=(EC₅₀ value of reference sample/EC₅₀ value of test sample)×100%

TABLE 2
Biological activity of PD-L1 terminus of test
samples measured by reporter gene assay.

| Sample | Biological activity | EC₅₀ (nmol/L) |
|---|---|---|
| PD-L1 antibody | 100% | 4.603 |
| hu5G11-hIgG1-TGFβ RII | 75.4% | 6.106 |
| PD-L1 antibody | 100% | 4.151 |
| M7824 | 72.5% | 5.724 |

Note:
The PD-L1 antibody (the reference sample) was a humanized 5G11-IgG1 antibody described in Pat. No., CN107001463 of heavy and light chain sequences set forth in SEQ ID NOs: 23 and 24 of the present application respectively. M7824 was an anti-PD-L1/TGF-β trap described in Pat. No., CN106103488 with heavy and light chain sequences set forth in SEQ ID NOs: 42 and 43, respectively. See Example 3 for the preparation method. Example 5 In-vitro Binding Activity Assay of Fusion Protein to PD-L1 and TGF-β by Enzyme-linked Immunosorbent Assay (ELISA).

In-Vitro Binding Assay of Fusion Protein to PD-L1

1) Coating: A PD-L1 recombinant protein (Sinobiological, 10084-H08H) was diluted with PBS to 2 μg/mL, immobilized on the 96-well plate at 100 μL/well, and incubated overnight at 4° C.

2) The plate was washed once with PBS+0.05% Tween 20, and a blocking buffer (PBS+3% BSA solution) was added at 250 μL/well for 1-2 h of blocking.

3) hu5G11-hIgG1-TGF-β RII was pre-diluted with a diluent (PBS+0.05% Tween 20+1% BSA) to about 4000 ng/mL, and then serially 4-fold diluted to the 7^{th} concentration (i.e., about 4000 ng/mL, about 1000 ng/mL, about 250 ng/mL, about 62.5 ng/mL, about 15.625 ng/mL, about 3.90625 ng/mL and about 0.9765625 ng/mL). After the PD-L1 recombinant protein was washed 3 times with PBS+0.05% Tween 20, the serially diluted fusion protein hu5G11-hIgG1-TGF-βRII and blank control were added at 100 μL/well, and incubated at 25° C. for 1-2 h.

4) The plate was washed 3 times with PBS+0.05% Tween 20, and an HRP-labeled goat anti-human secondary antibody (PE, NEF802001EA) diluted in a 1:3500 ratio was added at 100 μL/well. The plate was incubated at 25° C. for 1 h.

5) The plate was washed 3 times with PBS+0.05% Tween 20 before 3,3′,5,5′-tetramethylbenzidine (TMB) was added at 100 μL/well. The plate was incubated at 25° C. for 5 min in the dark. Finally, 1 M H2504 was added to terminate the reaction.

6) The absorbance was measured at 450 nm with a microplate reader (Thermo Scientific, Varioskan Flash). A curve was plotted with the average absorbance as the abscissa and the logarithmic concentration of hu5G11-hIgG1-TGFβ RII as the ordinate. Then the EC₅₀ of hu5G11-hIgG1-TGF-β RII was calculated.

In-Vitro Binding Assay of Fusion Protein to TGF-β

1) Coating: A TGF-β1 recombinant protein (Sinobiological, 10804-H08H) was diluted with PBS to 2 μg/mL, immobilized on the 96-well plate at 100 μL/well, and incubated overnight at 4° C.; the other procedures are as described above.

The results of the in-vitro binding assay of the fusion protein hu5G11-hIgG1-TGF-β RII in the examples of the present application to PD-L1 are shown in FIG. 3 and Table 3, while the results of the in-vitro binding assay of the fusion protein to TGF-β1 are shown in FIG. 4 and Table 3. The ELISA results demonstrate that the fusion protein retains the binding activity to PD-L1 and TGF-β.

TABLE 3
EC₅₀ of hu5G11-hIgG1-TGF-β RII binding
to PD-L1 and TGF-β by ELISA

| | PD-L1 terminus | TGF-β terminus |
|---|---|---|
| Sample | EC₅₀ (ng/ml) | EC₅₀ (ng/ml) |
| PD-L1/TGFβ RII | 34.31 | 67.03 |

### Example 6 Pharmacokinetic Analysis

1) Single-dose pharmacokinetic study: Cynomolgus monkeys as animal model were randomly divided into 3 groups of 6, half male and half female. The groups were infused intravenously with a single dose of 1, 10 and 60 mg/kg of fusion protein hu5G11-hIgG1-TGF-β RII. Whole blood samples were collected from the veins at 0 h pre-dose and at 1 min, 3 h, 8 h, 24 h, 48 h, 72 h, 120 h, 168 h, 216 h, 264 h, 336 h, 504 h and 672 h post-dose. The supernatant was separated, and parameters such as blood concentration were detected by ELISA. See Table 4 for the results.

2) Repeat-dose pharmacokinetic study: Cynomolgus monkeys as animal model, were randomly divided into 3 groups of 10, half male and half female. The monkeys were infused intravenously with 20, 60 and 200 mg/kg of fusion protein hu5G11-hIgG1-TGF-β RII once weekly for 4 weeks (5 doses in total). The blood was collected from the veins at 0 h before the first dose and at 1 min, 3 h, 8 h, 24 h, 48 h, 72 h, 120 h, and 168 h after the start of the first dose, at 0 h before the third and fourth doses and at 1 min after the start of the third and fourth doses, and at 0 h before the fifth dose and at 1 min, 3 h, 8 h, 24 h, 48 h, 72 h, 120 h, 168 h, 216 h, 264 h, 336 h, 504 h and 672 h after the start of the fifth dose. The supernatant was separated, and parameters such as blood concentration in the supernatant were detected by ELISA. See Table 5 for the results. The results in Table 5 indicate that no significant accumulation in the serum for hu5G11-hIgG1-TGF-β RII was observed.

The ELISA procedures are as follows:

1) Coating: A human PD-L1/B7-H1/CD274 Protein, Fc to (Sinobiological, LC11NO2402) was diluted with PBS to 1 μg/mL, immobilized on a 96-well plate at 100 μL/well, and incubated overnight at 4° C.;

2) The plate was washed 3 times with PBS+0.05% Tween 20, and a blocking buffer (a solution of PBS+0.05% Tween 20+1% BSA) was added at 300 μL/well at 25° C. for 2-3 h of blocking;

3) The plate was washed 3 times with PBS+0.05% Tween 20, and blank control samples, standard curve samples and test samples were added at 100 μL/well. The plate was incubated at 25° C. for 1-1.5 h;

4) The plate was washed 3 times with PBS+0.05% Tween 20. A human TGF-β RII biotinylated antibody (R&D, XL0519051) was diluted with a blocking buffer to a final concentration of 0.05 μg/mL and added to the plate at 100 μL/well. The plate was incubated at 25° C. for 1-1.5 h;

5) The plate was washed 3 times with PBS+0.05% Tween 20, and streptavidin-HRP diluted 1:200 with a blocking buffer was added at 100 μL/well. The plate was incubated at 25° C. for 30 min;

6) The plate was washed 3 times with PBS+0.05% Tween 20, and a TMB substrate was added at 100 μL/well. The plate was incubated in the dark at 25° C. for 5-10 min. Finally, 0.5 M H₂SO₄ was added at 100 μL/well to terminate the reaction.

7) The absorbance value at 450 nm was measured using a microplate reader.

TABLE 4
Single-dose pharmacokinetics for hu5G11-hIgG1-TGF-β RII

| | | Single dose | | | | |
|---|---|---|---|---|---|---|
| | | 1 mg/kg | | | 10 mg/kg | |
| Parameters | Unit | Female | Male | Mean | Female | Male |
| AUC_{INF}_{_{—}}_{obs} | h*μg/mL | 444 ± 164 | 460 ± 162 | 452 ± 146 | 8150 ± 311 | 8270 ± 558 |
| AUCₗₐₛₜ | h*μg/mL | 434 ± 170 | 447 ± 165 | 441 ± 150 | 8040 ± 341 | 7980 ± 551 |
| Cl_{_{—}}_{obs} | mL/h/kg | 2.47 ± 0.924 | 2.42 ± 1.04 | 2.45 ± 0.879 | 1.23 ± 0.0462 | 1.21 ± 0.0850 |
| Cₘₐₓ | μg/mL | 20.6 ± 1.51 | 19.2 ± 0.819 | 19.9 ± 1.33 | 214 ± 16.0 | 220 ± 13.5 |
| MRT_{INF}_{_{—}}_{obs} | h | 35.2 ± 12.0 | 43.1 ± 10.2 | 39.2 ± 10.8 | 66.7 ± 3.50 | 81.2 ± 8.44 |
| MRTₗₐₛₜ | h | 32.1 ± 13.5 | 38.1 ± 11.8 | 35.1 ± 11.8 | 63.5 ± 2.86 | 72.5 ± 6.54 |
| T_{1/2} | h | 26.5 ± 7.55 | 33.2 ± 5.97 | 29.8 ± 7.11 | 42.0 ± 7.06 | 36.7 ± 31.1 |
| Tₘₐₓ | h | 0.0167 ± 0.00 | 0.0167 ± 0.00 | 0.0167 ± 0.00 | 0.0167 ± 0.00 | 0.0167 ± 0.00 |
| Vₛₛ_{_{—}}_{obs} | mL/kg | 80.0 ± 2.87 | 97.3 ± 15.4 | 88.6 ± 13.7 | 81.8 ± 2.95 | 98.2 ± 8.43 |

| | | Single dose | | | |
|---|---|---|---|---|---|
| | | 10 mg/kg | 60 mg/kg | | |
| Parameters | Unit | Mean | Female | Male | Mean |
| AUC_{INF}_{_{—}}_{obs} | h*μg/mL | 8210 ± 410 | 44500 ± 10500 | 55200 ± 10200 | 49800 ± 10900 |
| AUCₗₐₛₜ | h*μg/mL | 8010 ± 411 | 44200 ± 11000 | 55000 ± 10400 | 49600 ± 11200 |
| Cl_{_{—}}_{obs} | mL/h/kg | 1.22 ± 0.0618 | 1.41 ± 0.383 | 1.11 ± 0.195 | 1.26 ± 0.316 |
| Cₘₐₓ | μg/mL | 217 ± 13.7 | 1360 ± 80.8 | 1260 ± 72.3 | 1310 ± 90.1 |
| MRT_{INF}_{_{—}}_{obs} | h | 74.0 ± 9.85 | 84.01 ± 1.6 | 109 ± 9.98 | 96.6 ± 16.9 |
| MRTₗₐₛₜ | h | 68.0 ± 6.69 | 80.1 ± 15.6 | 107 ± 7.39 | 93.8 ± 18.5 |
| T_{1/2} | h | 39.3 ± 20.4 | 56.1 ± 34.4 | 66.6 ± 38.9 | 61.3 ± 33.4 |
| Tₘₐₓ | h | 0.0167 ± 0.00 | 0.0167 ± 0.00 | 0.0167 ± 0.00 | 0.0167 ± 0.00 |
| Vₛₛ_{_{—}}_{obs} | mL/kg | 90.0 ± 10.6 | 116 ± 22.5 | 122 ± 29.0 | 119 ± 23.5 |

TABLE 5
Repeated-dose pharmacokinetics for hu5G11-hIgG1-TGF-β RII

| | | | Cₘₐₓ | AUC_{0-168 h} |
|---|---|---|---|---|
| Dosage | Time | Gender | (mg /mL) | (h*mg/mL) |
| 20 mg/kg | First dose | Female | 0.467 ± 0.0911 | 15.9 ± 3.97 |
| | | Male | 0.484 ± 0.116 | 17.4 ± 5.40 |
| | Last dose | Female | 0.282 ± 0.222 | 4.74 ± 6.53 |
| | | Male | 0.366 ± 0.284 | 15.3 ± 17.4 |
| 60 mg/kg | First dose | Female | 1.36 ± 0.126 | 43.9 ± 8.56 |
| | | Male | 1.43 ± 0.212 | 52.8 ± 13.0 |
| | Last dose | Female | 1.48 ± 0.398 | 45.8 ± 17.0 |
| | | Male | 1.25 ± 0.459 | 35.9 ± 19.7 |
| 200 mg/kg | First dose | Female | 5.96 ± 0.640 | 176 ± 31.2 |
| | | Male | 4.42 ± 0.772 | 168 ± 35.4 |
| | Last dose | Female | 5.50 ± 1.32 | 209 ± 86.7 |
| | | Male | 5.62 ± 1.11 | 210 ± 40.8 |

### Example 7 Inhibitory Effect of Fusion Protein on Mouse MC38 Graft Tumor

The DNA sequence of IgG1 was synthesized and cloned into an expression vector pcDNA3.1. Light and heavy chain expression vectors were co-transfected into ExpiCHO-S™ using an ExpiCHO transfection kit (Thermo Fisher, A29133). The cells were cultured in an incubator with 8% CO₂ at 37° C., and then the culture supernatant was purified using protein A filler as described in Example 2 to give IgG1. The heavy and light chain amino acid sequences of IgG1 are set forth in SEQ ID NO: 38 and SEQ ID NO: 39 of the present application.

The DNA sequence of IgG1-TGF-β RII was synthesized and cloned into an expression vector pcDNA3.1. Light and heavy chain expression vectors were co-transfected into ExpiCHO-S™ using an ExpiCHO transfection kit (Thermo Fisher, A29133). The cells were cultured in an incubator with 8% CO₂ at 37° C., and then the culture supernatant was purified using protein A filler as described in Example 2 to give IgG1-TGFβ RII. The heavy and light chain amino acid sequences of IgG1-TGF-β RII are set forth in SEQ ID NO: 40 and SEQ ID NO: 41 of the present application, respectively.

Humanized PD-L1 mice were subcutaneously inoculated with 3×10⁵ MC38/hPD-L1 cells/mouse to establish mouse colon cancer models. When tumors grew to 50-70 mm³, the mice were divided into groups of 10 according to the tumor volume, and then intravenously (IV) injected with 6 doses of hu5G11-hIgG1-TGF-β RII, IgG1 or IgG1-TGF-β RII once every two days in an injection volume of 0.1 mL/10 g body weight. The diameters of the tumors were measured twice weekly with a vernier caliper. The effect of the drug on tumor growth was examined based on the obtained T/C % or tumor growth inhibition TGI (%) calculated by the following formula. At the end of the experiment, at the study endpoint, or when the tumor volume reached 1500 mm³, the animals were sacrificed by CO₂ anesthesia and dissected to give the tumors. The tumors were photographed. The calculation formula of the tumor volume (V) is: V=½×a×b², wherein a and b represent the length and the width respectively; T/C (%)=(T−T₀)/(C−C₀)×100, wherein T and C are the tumor volumes of the treatment group and the isotype control group at the end of the experiment, and T₀ and C₀ are the tumor volumes of the treatment group and the isotype control group at the beginning of the experiment; tumor growth inhibition (TGI) (%)=100−T/C (%).

The results are shown in Table 6. hu5G11-hIgG1-TGF-β RII (at 3.7 and 12.3 mg/kg) has obvious inhibitory effects on the growth of MC38/hPD-L1 subcutaneous graft tumors with a tumor growth inhibition of 56% and 69% respectively, indicating a dosage dependence. The tumor growth inhibition of IgG1-TGF-β RII (2.3 mg/kg) to MC38/hPD-L1 subcutaneous graft tumors was 4%, and no significant efficacy was observed. Among them, the doses of the IgG1-TGF-β RII (2.3 mg/kg) and the hu5G11-hIgG1-TGF-β RII (3.7 mg/kg) were of the same order of magnitude and had no substantial difference. This confirms that the fusion protein of the present invention exhibits significantly higher tumor growth inhibition relative to fusion proteins having other compositions (i.e., fusion proteins comprising IgG1 and TGF-β RII). The tumor-bearing mice can well tolerate the above drug, without obvious symptoms such as weight loss.

TABLE 6
Efficacy of test samples on subcutaneous graft tumors of colon cancer cells MC38/hPD-L1

| | | | | | | Number of | |
|---|---|---|---|---|---|---|---|
| | | | | | | animals per | Number of |
| | Mean tumor | Mean tumor | T/C | Tumor growth | | group (at | animals per |
| | volume (mm³) | volume (mm³) | (%) | inhibition (%) | | beginning of | group (at end |
| Grouping | D0 | D17 | D17 | D17 | P value | study) | of study) |
| IgG1 | 57.3 ± 0.8 | 3344.4 ± 333.0 | — | — | — | 10 | 10 |
| 12.3 mg/kg | | | | | | | |
| hu5G11-hIgG1- | 56.8 ± 0.6 | 1513.1 ± 365.8 | 44 | 56 | 0.002 | 10 | 10 |
| TGFβ RII | | | | | | | |
| 3.7 mg/kg | | | | | | | |
| hu5G11-hIgG1- | 58.1 ± 1.9 | 1061.3 ± 330.7 | 31 | 69 | <0.001 | 10 | 10 |
| TGFβ RII | | | | | | | |
| 12.3 mg/kg | | | | | | | |
| IgG1-TGFb RII | 57.6 ± 0.6 | 3219.2 ± 279.0 | 96 | 4 | 0.776 | 10 | 10 |
| 2.3 mg/kg | | | | | | | |

Note:
The animals were randomized, the time for first administration was D0, and the treatment was given by intravenous injection (IV) once every two days in a total of 6 doses. P < 0.05, P < 0.01, P < 0.001 as compared to the IgG1 group (the isotype control) by T-test.

### Example 8 Cytokine Secretion Stimulated by Fusion Protein hu5G11-hIgG1-TGF-β RII Measured by Electrochemiluminescence

1) Liquid-phase method: PBMCs were adjusted with an RPMI1640 complete medium to a concentration of about 1-2×10⁶ cells/mL, and then added to a 96-well cell culture plate at 100 μL/well. IgG1 (SEQ ID NO: 38 and SEQ ID NO: 39, see Example 7 for the preparation method), LPS (SIGMA, L4391-1MG), and hu5G11-hIgG1-TGF-β RII were diluted with an RPMI1640 complete medium to give a 1000 μg/mL IgG 1 solution, a 10 μg/mL of LPS solution, and 100 μg/mL, 300 μg/mL and 1000 μg/mL of hu5G11-hIgG1-TGF-β RII solutions. The RPMI1640 complete medium was used as a negative control. 100 μL of prepared solutions was added into the 96-well cell culture plate and well mixed. The cells were cultured in a cell incubator with 5% CO₂ at 37° C. The cell supernatant was collected from the 96-well plate at 24 h and 48 h. The contents of cytokines IL-2, IL-4, IL-6, TNF-α and IFN-γ were measured using a V-PLEX Proinflammatory Panel 1 (human) kit (MSD, K15049D-2). The results are shown in Table 7.

2) Solid-phase method: IgG1 (SEQ ID NO: 38 and SEQ ID NO: 39, see Example 7 for the preparation method), LPS (SIGMA, L4391-1MG), and hu5G11-hIgG1-TGF-β RII were diluted with PBS to give a 500 μg/mL IgG 1 solution, a 5 μg/mL of LPS solution, and 50 μg/mL, 150 μg/mL and 500 μg/mL of hu5G11-hIgG1-TGF-β RII solutions; PBS was used as the negative control. The prepared solutions described above were added to the corresponding wells of the 96-well high-adsorption plate at 200 μL/well for coating at 37° C. for 2 h. The supernatant was discarded, and the cell culture plate was washed 3 times with PBS. PBMCs were adjusted with an RPMI1640 complete medium to a concentration of about 1-2×10⁶ cells/mL, added to a 96-well cell culture plate at 200 μL/well and incubated in a cell incubator at 37° C. with 5% CO₂. The cell supernatant was collected from the 96-well plate at 24 h and 48 h. The contents of cytokines IL-2, IL-4, IL-6, TNF-α and IFN-γ were measured using a V-PLEX Proinflammatory Panel 1 (human) kit (MSD, K15049D-2). The results are shown in Table 8.

TABLE 7
The results of cytokine secretion stimulated bv each test sample detected by liquid phase method

| | Sampling | | | | | |
|---|---|---|---|---|---|---|
| | points | IL-2 | IL-4 | IL-6 | TNF-α | IFN-γ |
| Grouping | (h) | (pg/ml) | (pg/ml) | (pg/ml) | (pg/ml) | (pg/ml) |
| RPMI1640 complete | 24 | 104.6 ± 137.7 | 1.3 ± 0.8 | 34.4 ± 22.3 | 14.0 ± 7.6 | 25.4 ± 9.7 |
| medium | 48 | 426.7 ± 621.7 | 1.1 ± 1.0 | 30.8 ± 34.7 | 23.6 ± 22.7 | 496.0 ± 174.5 |
| IgG1-1000 μg/mL | 24 | 164.2 ± 253.0 | 4.9 ± 0.4 | 623.0 ± 540.1 | 106.8 ± 70.6 | 93.7 ± 39.2 |
| | 48 | 226.0 ± 280.4 | 6.3 ± 2.3 | 2160.1 ± 3028.8 | 206.3 ± 229.7 | 1663.6 ± 1361.7 |
| LPS-10 μg/mL | 24 | 84.1 ± 59.8 | 36.9 ± 4.0 | 43920.0 ± 0 | 10357.2 ± 3119.2 | 2920.0 ± 0 |
| | 48 | 57.3 ± 19.3 | 43.3 ± 3.8 | 43920.0 ± 0 | 6254.8 ± 2317.6 | 2920.0 ± 0 |
| hu5G11-hIgG1-TGFβ | 24 | 193.2 ± 288.5 | 2.8 ± 0.8 | 203.0 ± 24.1 | 68.9 ± 13.6 | 82.1 ± 52.3 |
| RII-1000 μg/ml | 48 | 347.2 ± 531.1 | 2.1 ± 1.7 | 215.3 ± 39.0 | 82.2 ± 38.4 | 1588.6 ± 314.2 |
| hu5G11-hIgG1-TGFβ | 24 | 139.7 ± 126.3 | 3.6 ± 1.8 | 303.5 ± 237.0 | 62.0 ± 50.1 | 54.4 ± 20.4 |
| RII-300 μg/ml | 48 | 357.9 ± 484.2 | 4.0 ± 1.7 | 485.6 ± 341.9 | 64.4 ± 27.3 | 839.6 ± 667.8 |
| hu5G11-hIgG1-TGFβ | 24 | 144.4 ± 225.9 | 2.1 ± 1.2 | 127.2 ± 40.2 | 25.6 ± 3.9 | 54.3 ± 57.0 |
| RII-100 μg/ml | 48 | 315.4 ± 407.4 | 2.7 ± 1.4 | 195.1 ± 67.5 | 40.2 ± 18.6 | 1229.9 ± 1466.5 |

TABLE 8
The results of cytokine secretion stimulated by each test sample detected by solid phase method

| | Sampling | IL-2 | IL-4 | IL-6 | TNF-α | IFN-γ |
|---|---|---|---|---|---|---|
| Grouping | time (h) | (pg/ml) | (pg/ml) | (pg/ml) | (pg/ml) | (pg/ml) |
| PBS | 24 | 113.3 ± 150.1 | 1.0 ± 0.9 | 62.6 ± 20.2 | 27.9 ± 7.4 | 40.2 ± 7.1 |
| | 48 | 281.7 ± 384.2 | 1.2 ± 0.7 | 99.0 ± 79.8 | 43.7 ± 17.4 | 516.4 ± 248.8 |
| IgG1-500 μg/mL | 24 | 86.8 ± 75.2 | 6.1 ± 0.2 | 894.3 ± 407.1 | 955.5 ± 562.2 | 58.0 ± 24.2 |
| | 48 | 210.2 ± 202.7 | 6.1 ± 0.4 | 891.0 ± 287.5 | 808.3 ± 292.7 | 435.4 ± 630.9 |
| LPS-5 μg/mL | 24 | 49.7 ± 15.3 | 48.8 ± 12.1 | 43920.0 ± 0 | 10700.0 ± 3719.3 | 2920.0 ± 0 |
| | 48 | 43.7 ± 1.4 | 57.7 ± 9.2 | 43920.0 ± 0 | 10658.6 ± 4817.8 | 2920.0 ± 0 |
| hu5G11-hIgG1-TGFβ | 24 | 140.2 ± 141.7 | 7.1 ± 1.1 | 1769.6 ± 1294.3 | 1125.5 ± 1122.7 | 162.1 ± 142.0 |
| RII -500 μg/ml | 48 | 308.7 ± 359.2 | 6.8 ± 1.0 | 965.9 ± 229.7 | 701.9 ± 541.8 | 716.8 ± 1063.5 |
| hu5G11-hIgG1-TGFβ | 24 | 136.3 ± 157.4 | 6.0 ± 1.8 | 1747.8 ± 2238.3 | 1118.1 ± 1379.8 | 195.0 ± 260.2 |
| RII -150 μg/ml | 48 | 240.9 ± 263.4 | 5.4 ± 0.8 | 525.7 ± 216.5 | 459.3 ± 317.3 | 644.3 ± 835.4 |
| hu5G11-hIgG1-TGFβ | 24 | 160.2 ± 193.3 | 6.5 ± 2.1 | 1266.4 ± 1669.4 | 842.1 ± 1063.9 | 161.2 ± 177.0 |
| RII -50 μg/ml | 48 | 393.6 ± 454.6 | 6.0 ± 1.1 | 430.3 ± 243.6 | 507.3 ± 417.0 | 539.7 ± 678.8 |

As can be seen from the results in the above table, the fusion protein of the present application exhibits a low probability of causing a cytokine storm. Therefore, the subject substantially has no risk of systemic inflammation caused by overactivating the immune system after administration.

### Example 9 Toxicity Assay of Fusion Protein in Cynomolgus Monkeys

The anal temperature was detected using a Temp-14 thermistor thermometer. The respiratory rate and an II-lead electrocardiogram (ECG) were detected using noninvasive physiological signal telemetry system for large animals. The blood pressure (the systolic pressure, the diastolic pressure, and the mean arterial pressure) was measured using an noninvasive sphygmomanometer. An electrocardiogram waveform was qualitatively analyzed, and the stable and continuous electrocardiogram within a 30-second period at each time point is analyzed using ecgAUTO software. The parameters included heart rate, R-R interval, P-wave time, P-R interval, QRS wave time, Q-T interval and corrected Q-T interval.

Single-dose toxicity: In this test, 6 cynomolgus monkeys were divided into two groups of 3, including male and female. The monkeys were infused intravenously with a single dose of 300 or 1000 mg/kg of hu5G11-hIgG1-TGF-β RII, and observed for 14 days. During the test, indicators such as general observation, body weight, food intake, body temperature, II-lead ECG and blood pressure, hematology, blood biochemistry, and urine were detected. Gross anatomical observation was performed at the end of test. No death or near-death was observed during the test, and no abnormality was found in all indicators.

Repeat-dose toxicity: In this test, 40 cynomolgus monkeys were divided into 4 groups of 10, i.e., the blank control group and the treatment groups receiving 20, 60 and 200 mg/kg of hu5G11-hIgG1-TGF-β RII, half male and half female. The drug was administered once a week for 4 weeks (5 doses in total). After discontinuation, the monkeys were observed for 6 weeks during recovery. Toxicokinetic parameters of the repeated intravenous injection toxicity study are shown in Table 5 of Example 6.

At the end of treatment, the 60 mg/kg and 200 mg/kg treatment groups showed mild anemia that mainly featured decreased RBC, HGB and HCT, and increased RET and RET %. The 200 mg/kg treatment group also showed mild increase in the proportions of erythron and orthochromatic normoblasts in bone marrow smears. Histopathological examination showed that the bone marrow and the spleen had no relevant changes. By the end of the recovery period, the HGB and HCT in one female monkey in the 200 mg/kg treatment group still had no obvious recovery, and examination of the red blood cell morphology showed a decrease in hemoglobin content. The indicators of other cynomolgus monkeys described above all recovered to different extents.

At the end of treatment, slight mononuclear cell infiltration in thyroid gland was seen in the 20, 60 and 200 mg/kg treatments groups, slight to mild mononuclear cell infiltration in the kidney was seen in the 60 and 200 mg/kg treatment groups, while slight mononuclear cell infiltration in the meninx was seen in the 20 and 200 mg/kg treatment groups. By the end of the recovery period, except that the meningeal lesion in the 200 mg/kg treatment group did not recover, the organ lesions of other cynomolgus monkeys showed certain degrees of recovery. In addition, no obvious abnormal change was observed in the cynomolgus monkeys of all the groups in the aspect of general observation, body weight, food intake, body temperature, II-lead ECG, respiratory rate and blood pressure, blood biochemistry, urine, ophthalmologic examination, bone marrow smear, immunoglobulin, complement, circulating immune complex, lymphocyte subpopulation and cytokines.

As can be seen, the fusion protein of the present application shows low acute toxicity and long-term toxicity. Therefore, it is expected that the fusion protein will show good safety in clinic use.

According to the content disclosed in the present application, the compositions and methods of the present application have been described in terms of preferred embodiments. However, it will be apparent to those skilled in the art that variations may be applied to the compositions and/or methods and the steps or the sequence of steps of the methods described herein without departing from the concept, spirit and scope of the present application. The disclosed contents of all documents cited herein are hereby incorporated by reference to the extent that they provide exemplary, procedural and other details supplementary to those described herein.

1. A protein comprising: (a) at least one heavy chain variable domain and at least one light chain variable domain of an antibody binding to human programmed death-ligand 1 (PD-L1); and (b) human TGF-β RII or a TGF-β binding fragment thereof,
wherein, in the antibody, a heavy chain CDR1 sequence comprises an amino acid sequence set forth in SEQ ID NO: 1 or SEQ ID NO: 15, a heavy chain CDR2 sequence comprises an amino acid sequence set forth in SEQ ID NO: 2 or SEQ ID NO: 16, and a heavy chain CDR3 sequence comprises an amino acid sequence set forth in SEQ ID NO: 3 or SEQ ID NO: 17; and a light chain CDR1 sequence comprises an amino acid sequence set forth in SEQ ID NO: 4 or SEQ ID NO: 18, a light chain CDR2 sequence comprises an amino acid sequence set forth in SEQ ID NO: 5 or SEQ ID NO: 19, and a light chain CDR3 sequence comprises an amino acid sequence set forth in SEQ ID NO: 6 or SEQ ID NO: 20.

2. The protein according to claim 1, wherein the heavy chain variable domain comprises an amino acid sequence set forth in SEQ ID NO: 7 or SEQ ID NO: 21, and the light chain variable domain comprises an amino acid sequence set forth in SEQ ID NO: 8 or SEQ ID NO: 22.

3. The protein according to claim 1, wherein the heavy chain variable domain comprises an amino acid sequence set forth in SEQ ID NO: 7, and the light chain variable domain comprises an amino acid sequence set forth in SEQ ID NO: 8.

4. The protein according to claim 1, wherein the heavy chain variable domain comprises an amino acid sequence set forth in SEQ ID NO: 21, and the light chain variable domain comprises an amino acid sequence set forth in SEQ ID NO: 22.

5. The protein according to claim 1, wherein the antibody comprises: a heavy chain amino acid sequence having at least 95% identity to an amino acid sequence set forth in SEQ ID NO: 9, SEQ ID NO: 23, SEQ ID NO: 29 or SEQ ID NO: 32; and a light chain amino acid sequence having at least 95% identity to an amino acid sequence set forth in SEQ ID NO: 10 or SEQ ID NO: 24.

6. The protein according to claim 1, wherein the human TGF-β RII or the TGF-β binding fragment thereof comprises an amino acid sequence having at least 80% identity to an amino acid sequence set forth in SEQ ID NO: 36.

7. The protein according to claim 1, wherein the protein further comprises a linker peptide linking a C terminus of the antibody or the antigen binding fragment thereof to an N terminus of the human TGF-β RII or the TGF-β binding fragment thereof.

8. The protein according to claim 7, wherein the linker peptide is (G₄S)ₓG, and x is an integer of 3-6.

9. A polynucleotide sequence encoding the protein according to claim 1.

10. The polynucleotide sequence according to claim 9, wherein the polynucleotide sequence comprises: a nucleic acid sequence set forth in SEQ ID NO: 12, a nucleic acid sequence set forth in SEQ ID NO: 26, a nucleic acid sequence set forth in SEQ ID NO: 31 or a nucleic acid sequence set forth in SEQ ID NO: 34, and a nucleic acid sequence set forth in SEQ ID NO: 14 or a nucleic acid sequence set forth in SEQ ID NO: 28.

11-12. (canceled)

13. A method for treating cancer, inhibiting tumor growth, or enhancing an anti-tumor response, comprising administering to a subject in need thereof the protein according to claim 1.

14. The method according to claim 13, wherein the protein is administered as monotherapy or in combination with radiotherapy, chemotherapy, surgery, biologics, or chemicals.

15. The method according to claim 13, wherein the cancer or tumor is selected from the group consisting of: lung adenocarcinoma, mucinous adenocarcinoma, low-grade brain neuroglioma, glioblastoma multiforme, mesothelioma, melanoma, thyroid cancer, renal cancer, liver cancer, acute myeloid leukemia, esophageal adenocarcinoma, lymphoma, non-small cell lung cancer, metastatic non-small cell lung cancer, advanced or recurrent non-small cell lung cancer, refractory non-small cell lung cancer after chemotherapy, metastatic non-squamous non-small cell lung cancer, advanced or recurrent non-squamous non-small cell lung cancer, unresectable advanced non-small cell lung cancer, occult non-small cell lung cancer, breast cancer, metastatic breast cancer, triple-negative breast cancer, advanced or recurrent breast cancer, locally recurrent breast cancer, inflammatory breast cancer, pancreatic ductal adenocarcinoma, metastatic pancreatic cancer, locally advanced unresectable pancreatic cancer, recurrent pancreatic cancer, prostate cancer, advanced or metastatic prostate cancer, locally advanced prostate cancer, castration-resistant prostate cancer, recurrent prostate cancer after castration, localized prostate cancer, progressive prostate cancer, colorectal cancer, rectal adenocarcinoma, large intestinal cancer, metastatic colorectal cancer, advanced or recurrent colon cancer, advanced or recurrent rectal cancer, locally recurrent rectal cancer, locally recurrent colon cancer, gastric adenocarcinoma, gastric cancer, unresectable gastric cancer, metastatic gastric cancer, locally advanced or recurrent gastric cancer, gastrointestinal stromal tumor, biliary tract cancer, bile duct cancer, gallbladder cancer, unresectable or metastatic biliary tract cancer, unresectable or metastatic bile duct cancer, unresectable or metastatic gallbladder cancer, penile cancer, anal cancer, vaginal cancer, cervical cancer, locally advanced cervical cancer, recurrent cervical cancer, metastatic cervical cancer, metastatic penile cancer, advanced or recurrent vaginal squamous cell carcinoma, advanced or recurrent vaginal adenocarcinoma, uterine corpus endometrioid carcinoma, bladder cancer, human papillomavirus infection, head and neck cancer, recurrent or metastatic head and neck cancer, hypopharyngeal cancer, laryngeal cancer, oral cancer, nasopharyngeal carcinoma, oropharyngeal cancer, pharyngolaryngeal cancer, paranasal sinus and nasal cavity cancer, and salivary gland cancer.

16. The protein according to claim 8, wherein the linker peptide has an amino acid sequence set forth in SEQ ID NO: 37.

17. The polynucleotide sequence according to claim 10, wherein the polynucleotide sequence comprises: the nucleic acid sequences set forth in SEQ ID NO: 34 and SEQ ID NO: 28, or the nucleic acid sequences set forth in SEQ ID NO: 31 and SEQ ID NO: 14.

18. The protein according to claim 5, wherein the antibody comprises:
a heavy chain amino acid sequence as set forth in SEQ ID NO: 9; and a light chain amino acid sequence as set forth in SEQ ID NO: 10;
a heavy chain amino acid sequence as set forth in SEQ ID NO: 23; and a light chain amino acid sequence as set forth in SEQ ID NO: 24;
a heavy chain amino acid sequence as set forth in SEQ ID NO: 29; and a light chain amino acid sequence as set forth in SEQ ID NO: 10; or
a heavy chain amino acid sequence as set forth in SEQ ID NO: 32; and a light chain amino acid sequence as set forth in SEQ ID NO: 24.

19. The protein according to claim 1, wherein the protein comprises: (1) two identical first polypeptides with an amino acid sequence having at least 80% identity to an amino acid sequence set forth in SEQ ID NO: 11 or SEQ ID NO: 25, or with an amino acid sequence having at least 80% identity to an amino acid sequence set forth in SEQ ID NO: 30 or SEQ ID NO: 33; and (2) two identical second polypeptides with an amino acid sequence having at least 80% identity to an amino acid sequence set forth in SEQ ID NO: 13 or SEQ ID NO: 27.

20. The protein according to claim 19, wherein the protein comprises: (1) two identical first polypeptides with an amino acid sequence having at least 80% identity to the amino acid sequence set forth in SEQ ID NO: 33; and (2) two identical second polypeptides with an amino acid sequence having at least 80% identity to the amino acid sequence set forth in SEQ ID NO: 27.


SEQUENCE LISTING (43 sequences)

SEQ ID NO: 1 - protein, 5 aa, Mus sp.
SYGMS

SEQ ID NO: 2 - protein, 16 aa, Mus sp.
SISSGGSTYYPDSVKG

SEQ ID NO: 3 - protein, 8 aa, Mus sp.
GYDSGFAY

SEQ ID NO: 4 - protein, 14 aa, Mus sp.
ASQSVSTSSSSFMH

SEQ ID NO: 5 - protein, 7 aa, Mus sp.
YASNLES

SEQ ID NO: 6 - protein, 9 aa, Mus sp.
QHSWEIPYT

SEQ ID NO: 7 - protein, 116 aa, Artificial sequence
  Heavy chain variable domain of PD-L1 antibody
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSS

SEQ ID NO: 8 - protein, 111 aa, Artificial sequence
  Light chain variable domain of PD-L1 antibody
DIVLTQSPASLAVSPGQRATITCRASQSVSTSSSSFMHWYQQKPGQPPKLLIKYASNLESGVPARFSGSGSGTDFTLTINPVEANDTANYYCQHSWEIPYTFGQGTKLEIK

SEQ ID NO: 9 - protein, 446 aa, Artificial sequence
  Heavy chain of PD-L1 antibody
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, Artificial sequence
  Light chain of PD-L1 antibody
DIVLTQSPASLAVSPGQRATITCRASQSVSTSSSSFMHWYQQKPGQPPKLLIKYASNLESGVPARFSGSGSGTDFTLTINPVEANDTANYYCQHSWEIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 603 aa, Artificial sequence
  Heavy chain of anti-PD-L1-TGF-beta RII fusion protein
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 12 - DNA, 1809 bp, Artificial sequence
  Nucleic acid sequence for encoding heavy chain of anti-PD-L1-TGF-beta RII fusion protein
gaggtgcagctggtcgagtcaggaggggggctggtcaagccaggagggtcactgcgactgagctgcgcagcttccgggttcatctttaggtcttatggcatgagttgggtgcgccaggcaccagggaaaggactggagtgggtcgcttcaatcagctccggaggcagcacttactatcctgactccgtgaagggccggttcaccatttctagagataacgccaaaaatagtctgtacctgcagatgaactctctgcgagcagaagacacagccgtctacgattgtgctagaggatatgacagcggctttgcatactggggccaggggaccctggtgacagtctcgagcgcctccactaagggcccatccgtgttccctctggcaccctccagcaagagcacaagcggaggcaccgccgcactgggctgcctcgtgaaggactacttcccagaacccgtgaccgtcagctggaatagcggcgctctgaccagcggagtccacactttccccgcagtgctgcagtccagcggcctgtacagcctgagcagcgtggtcactgtgccaagcagcagcctgggcactcagacctacatctgcaacgtcaaccacaagcccagcaacacaaaggtggacaagaaggtcgagcccaagtcctgcgataagacccacacctgccctccatgtcccgcccccgagctgctgggaggacccagcgtcttcctgtttccccccaagccaaaggacaccctgatgatcagcaggacccccgaagtgacctgcgtcgtggtggccgtgagccacgaagatcccgaggtgaagttcaactggtacgtggacggcgtggaagtgcacaacgccaagacaaaacccagggaggagcagtataacagcacctacagggtcgtgagcgtcctgaccgtgctgcaccaagactggctgaacggcaaggagtataagtgcaaggtgagcaacaaggcactgcccgcccccatcgagaagaccatttccaaggccaaggggcaacctagggagccacaggtctacactctgccccctagcagggacgagctgaccaagaaccaggtctccctgacttgcctggtgaaggggttttatcccagcgacatcgccgtcgagtgggagagcaatggccagcccgaaaacaactacaagaccacaccccctgtgctggacagcgacggcagcttctttctgtatagcaaactgacagtggataagagcagatggcagcagggcaacgtgttctcctgctccgtgatgcacgaggccctgcacaatcactacacccagaagtccctgagcctgtcccccggaaaaggaggaggaggatctggaggaggcggcagcggcggaggaggaagtggaggaggcggatccggcatccctccccacgtgcagaagagcgtgaataacgacatgatcgtgaccgacaataacggcgctgtgaagttccctcagctgtgcaagttctgcgatgtgcggttctccacctgcgacaatcagaagagctgcatgagcaactgcagcatcacctccatctgcgagaagcctcaggaggtgtgtgtggccgtgtggcggaagaatgacgagaatatcaccctggagaccgtgtgccacgaccccaagctgccttatcacgatttcatcctggaggacgccgctagccccaagtgcatcatgaaggagaagaagaagcccggcgagaccttcttcatgtgtagctgtagcagcgatgagtgcaacgataatatcatctttagcgaggagtataacaccagcaatcccgat

SEQ ID NO: 13 - protein, 218 aa, Artificial sequence; identical to SEQ ID NO: 10
  Light chain of anti-PD-L1-TGF-beta RII fusion protein

SEQ ID NO: 14 - DNA, 654 bp, Artificial sequence
  Nucleic acid sequence for encoding light chain of anti-PD-L1-TGF-beta RII fusion protein
gacattgtgctgactcagagccccgcttcactggcagtgtctccagggcagcgggcaaccatcacatgcagagcctcacagagcgtctccaccagctcctctagtttcatgcactggtaccagcagaagcccggacagccccctaagctgctgatcaaatatgctagcaacctggagtccggcgtgccagccaggttctctggcagtgggtcaggaaccgactttactctgaccattaatcccgtcgaagccaacgatacagctaattactattgtcagcattcctgggagatcccttacacatttggccaggggactaagctggagatcaagcgtacggtggccgcaccaagcgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagctttaacagaggcgagtgc

SEQ ID NO: 15 - protein, 5 aa, Mus sp.
TYGVH

SEQ ID NO: 16 - protein, 16 aa, Mus sp.
VIWRGVTTDYNAAFMS

SEQ ID NO: 17 - protein, 8 aa, Mus sp.
LGFYAMDY

SEQ ID NO: 18 - protein, 11 aa, Mus sp.
KASQSVSNDVA

SEQ ID NO: 19 - protein, 7 aa, Mus sp.
YAANRYT

SEQ ID NO: 20 - protein, 9 aa, Mus sp.
QQDYTSPYT

SEQ ID NO: 21 - protein, 116 aa, Artificial sequence
  Heavy chain variable domain of PD-L1 antibody
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSS

SEQ ID NO: 22 - protein, 107 aa, Artificial sequence
  Light chain variable domain of PD-L1 antibody
DIQMTQSPSSLSASVGDRVTITCKASQSVSNDVAWYQQKPGKAPKLLIYYAANRYTGVPDRFSGSGYGTDFTFTISSLQPEDIATYFCQQDYTSPYTFGQGTKLEIK

SEQ ID NO: 23 - protein, 446 aa, Artificial sequence
  Heavy chain of PD-L1 antibody
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial sequence
  Light chain of PD-L1 antibody
DIQMTQSPSSLSASVGDRVTITCKASQSVSNDVAWYQQKPGKAPKLLIYYAANRYTGVPDRFSGSGYGTDFTFTISSLQPEDIATYFCQQDYTSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 603 aa, Artificial sequence
  Heavy chain of anti-PD-L1-TGF-beta RII fusion protein
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 26 - DNA, 1809 bp, Artificial sequence
  Nucleic acid sequence for encoding heavy chain of anti-PD-L1-TGF-beta RII fusion protein
cagatcacactgaaagaaagcggccctaccctggtcaagccaactcagaccctgacactgacttgcaccgtgtctgggttctctctgagtacatacggagtccactggatcaggcagccccctggcaaagctctggagtggctgggagtgatttggcggggcgtcaccacagactataacgccgcttttatgtcaagactgacaatcactaaggataacagcaaaaatcaggtggtcctgaccatgaacaatatggaccccgtggataccgcaacatactattgtgcccggctggggttctacgccatggactattggggccaggggactctggtgaccgtctcgagcgcctccactaagggaccatccgtgttccctctggcaccctccagcaagagcacaagcggaggcaccgccgcactgggctgcctcgtgaaggactacttcccagaacccgtgaccgtcagctggaatagcggcgctctgaccagcggagtccacactttccccgcagtgctgcagtccagcggcctgtacagcctgagcagcgtggtcactgtgccaagcagcagcctgggcactcagacctacatctgcaacgtcaaccacaagcccagcaacacaaaggtggacaagaaggtcgagcccaagtcctgcgataagacccacacctgccctccatgtcccgcccccgagctgctgggaggacccagcgtcttcctgtttccccccaagccaaaggacaccctgatgatcagcaggacccccgaagtgacctgcgtcgtggtggccgtgagccacgaagatcccgaggtgaagttcaactggtacgtggacggcgtggaagtgcacaacgccaagacaaaacccagggaggagcagtataacagcacctacagggtcgtgagcgtcctgaccgtgctgcaccaagactggctgaacggcaaggagtataagtgcaaggtgagcaacaaggcactgcccgcccccatcgagaagaccatttccaaggccaaggggcaacctagggagccacaggtctacactctgccccctagcagggacgagctgaccaagaaccaggtctccctgacttgcctggtgaaggggttttatcccagcgacatcgccgtcgagtgggagagcaatggccagcccgaaaacaactacaagaccacaccccctgtgctggacagcgacggcagcttctttctgtatagcaaactgacagtggataagagcagatggcagcagggcaacgtgttctcctgctccgtgatgcacgaggccctgcacaatcactacacccagaagtccctgagcctgtcccccggaaaaggaggaggaggatctggaggaggcggcagcggcggaggaggaagtggaggaggcggatccggcatccctccccacgtgcagaagagcgtgaataacgacatgatcgtgaccgacaataacggcgctgtgaagttccctcagctgtgcaagttctgcgatgtgcggttctccacctgcgacaatcagaagagctgcatgagcaactgcagcatcacctccatctgcgagaagcctcaggaggtgtgtgtggccgtgtggcggaagaatgacgagaatatcaccctggagaccgtgtgccacgaccccaagctgccttatcacgatttcatcctggaggacgccgctagccccaagtgcatcatgaaggagaagaagaagcccggcgagaccttcttcatgtgtagctgtagcagcgatgagtgcaacgataatatcatctttagcgaggagtataacaccagcaatcccgat

SEQ ID NO: 27 - protein, 214 aa, Artificial sequence; identical to SEQ ID NO: 24
  Light chain of anti-PD-L1-TGF-beta RII fusion protein

SEQ ID NO: 28 - DNA, 642 bp, Artificial sequence
  Nucleic acid sequence for encoding light chain of anti-PD-L1-TGF-beta RII fusion protein
gacatccagatgactcagtctccaagcagcctgtctgcatctgtgggggacagggtcaccatcacatgcaaagcatctcagagtgtgtcaaacgatgtcgcctggtaccagcagaagcccggaaaagctcctaagctgctgatttactatgccgctaatcggtacactggcgtgccagacagattcagcggatccggatatggaaccgatttcacttttaccatcagctccctgcagccagaggacattgccacatatttctgtcagcaggattacacaagcccctatacttttggccaggggaccaaactggaaatcaagcgtacggtggccgcaccaagcgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagctttaacagaggcgagtgc

SEQ ID NO: 29 - protein, 446 aa, Artificial sequence
  Heavy chain of PD-L1 antibody
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 30 - protein, 603 aa, Artificial sequence
  Heavy chain of anti-PD-L1-TGF-beta RII fusion protein
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 31 - DNA, 1809 bp, Artificial sequence
  Nucleic acid sequence for encoding heavy chain of anti-PD-L1-TGF-beta RII fusion protein
gaggtgcagctggtcgagtcaggaggggggctggtcaagccaggagggtcactgcgactgagctgcgcagcttccgggttcatctttaggtcttatggcatgagttgggtgcgccaggcaccagggaaaggactggagtgggtcgcttcaatcagctccggaggcagcacttactatcctgactccgtgaagggccggttcaccatttctagagataacgccaaaaatagtctgtacctgcagatgaactctctgcgagcagaagacacagccgtctacgattgtgctagaggatatgacagcggctttgcatactggggccaggggaccctggtgacagtctcgagcgcctccactaagggcccatccgtgttccctctggcaccctccagcaagagcacaagcggaggcaccgccgcactgggctgcctcgtgaaggactacttcccagaacccgtgaccgtcagctggaatagcggcgctctgaccagcggagtccacactttccccgcagtgctgcagtccagcggcctgtacagcctgagcagcgtggtcactgtgccaagcagcagcctgggcactcagacctacatctgcaacgtcaaccacaagcccagcaacacaaaggtggacaagaaggtcgagcccaagtcctgcgataagacccacacctgccctccatgtcccgcccccgagctgctgggaggacccagcgtcttcctgtttccccccaagccaaaggacaccctgatgatcagcaggacccccgaagtgacctgcgtcgtggtggccgtgagccacgaagatcccgaggtgaagttcaactggtacgtggacggcgtggaagtgcacaacgccaagacaaaacccagggaggagcagtataacagcacctacagggtcgtgagcgtcctgaccgtgctgcaccaagactggctgaacggcaaggagtataagtgcaaggtgagcaacaaggcactgcccgcccccatcgagaagaccatttccaaggccaaggggcaacctagggagccacaggtctacactctgccccctagcagggacgagctgaccaagaaccaggtctccctgacttgcctggtgaaggggttttatcccagcgacatcgccgtcgagtgggagagcaatggccagcccgaaaacaactacaagaccacaccccctgtgctggacagcgacggcagcttctttctgtatagcaaactgacagtggataagagcagatggcagcagggcaacgtgttctcctgctccgtgatgcacgaggccctgcacaatcactacacccagaagtccctgagcctgtcccccggagccggaggaggaggatctggaggaggcggcagcggcggaggaggaagtggaggaggcggatccggcatccctccccacgtgcagaagagcgtgaataacgacatgatcgtgaccgacaataacggcgctgtgaagttccctcagctgtgcaagttctgcgatgtgcggttctccacctgcgacaatcagaagagctgcatgagcaactgcagcatcacctccatctgcgagaagcctcaggaggtgtgtgtggccgtgtggcggaagaatgacgagaatatcaccctggagaccgtgtgccacgaccccaagctgccttatcacgatttcatcctggaggacgccgctagccccaagtgcatcatgaaggagaagaagaagcccggcgagaccttcttcatgtgtagctgtagcagcgatgagtgcaacgataatatcatctttagcgaggagtataacaccagcaatcccgat

SEQ ID NO: 32 - protein, 446 aa, Artificial sequence
  Heavy chain of PD-L1 antibody
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 33 - protein, 603 aa, Artificial sequence
  Heavy chain of anti-PD-L1-TGF-beta RII fusion protein
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - DNA, 1809 bp, Artificial sequence
  Nucleic acid sequence for encoding heavy chain of anti-PD-L1-TGF-beta RII fusion protein
cagatcacactgaaagaaagcggccctaccctggtcaagccaactcagaccctgacactgacttgcaccgtgtctgggttctctctgagtacatacggagtccactggatcaggcagccccctggcaaagctctggagtggctgggagtgatttggcggggcgtcaccacagactataacgccgcttttatgtcaagactgacaatcactaaggataacagcaaaaatcaggtggtcctgaccatgaacaatatggaccccgtggataccgcaacatactattgtgcccggctggggttctacgccatggactattggggccaggggactctggtgaccgtctcgagcgcctccactaagggaccatccgtgttccctctggcaccctccagcaagagcacaagcggaggcaccgccgcactgggctgcctcgtgaaggactacttcccagaacccgtgaccgtcagctggaatagcggcgctctgaccagcggagtccacactttccccgcagtgctgcagtccagcggcctgtacagcctgagcagcgtggtcactgtgccaagcagcagcctgggcactcagacctacatctgcaacgtcaaccacaagcccagcaacacaaaggtggacaagaaggtcgagcccaagtcctgcgataagacccacacctgccctccatgtcccgcccccgagctgctgggaggacccagcgtcttcctgtttccccccaagccaaaggacaccctgatgatcagcaggacccccgaagtgacctgcgtcgtggtggccgtgagccacgaagatcccgaggtgaagttcaactggtacgtggacggcgtggaagtgcacaacgccaagacaaaacccagggaggagcagtataacagcacctacagggtcgtgagcgtcctgaccgtgctgcaccaagactggctgaacggcaaggagtataagtgcaaggtgagcaacaaggcactgcccgcccccatcgagaagaccatttccaaggccaaggggcaacctagggagccacaggtctacactctgccccctagcagggacgagctgaccaagaaccaggtctccctgacttgcctggtgaaggggttttatcccagcgacatcgccgtcgagtgggagagcaatggccagcccgaaaacaactacaagaccacaccccctgtgctggacagcgacggcagcttctttctgtatagcaaactgacagtggataagagcagatggcagcagggcaacgtgttctcctgctccgtgatgcacgaggccctgcacaatcactacacccagaagtccctgagcctgtcccccggagccggaggaggaggatctggaggaggcggcagcggcggaggaggaagtggaggaggcggatccggcatccctccccacgtgcagaagagcgtgaataacgacatgatcgtgaccgacaataacggcgctgtgaagttccctcagctgtgcaagttctgcgatgtgcggttctccacctgcgacaatcagaagagctgcatgagcaactgcagcatcacctccatctgcgagaagcctcaggaggtgtgtgtggccgtgtggcggaagaatgacgagaatatcaccctggagaccgtgtgccacgaccccaagctgccttatcacgatttcatcctggaggacgccgctagccccaagtgcatcatgaaggagaagaagaagcccggcgagaccttcttcatgtgtagctgtagcagcgatgagtgcaacgataatatcatctttagcgaggagtataacaccagcaatcccgat

SEQ ID NO: 35 - protein, 567 aa, Homo sapiens
  Human TGF-beta RII isoform B precursor polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 36 - protein, 136 aa, Homo sapiens
  Human TGF-beta RII extracellular domain polypeptide
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 37 - protein, 21 aa, Artificial sequence
  Linker peptide
GGGGSGGGGSGGGGSGGGGSG

SEQ ID NO: 38 - protein, 446 aa, SArtificial sequence
  Heavy chain of IgG1
EVQLEQSGAELMKPGASVKISCKATGYTFTTYWIEWIKQRPGHSLEWIGEILPGSDSTYYNEKVKGKVTFTADASSNTAYMQLSSLTSEDSAVYYCARGDGFYVYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, Artificial sequence
  Light chain of IgG1
DIELTQSPATLSVTPGDSVSLSCRASQSISNNLHWYQQKSHESPRLLIKYTSQSMSGIPSRFSGSGSGTDFTLSINSVETEDFGVYFCQQSGSWPRTFGGGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 603 aa, Artificial sequence
  Heavy chain of IgG1-TGF-beta RII
EVQLEQSGAELMKPGASVKISCKATGYTFTTYWIEWIKQRPGHSLEWIGEILPGSDSTYYNEKVKGKVTFTADASSNTAYMQLSSLTSEDSAVYYCARGDGFYVYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 41 - protein, 214 aa, Artificial sequence; identical to SEQ ID NO: 39
  Light chain of IgG1-TGF-beta RII

SEQ ID NO: 42 - protein, 607 aa, Artificial Sequence
  Heavy chain of M7824
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 43 - protein, 216 aa, Artificial Sequence
  Light chain of M7824
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS